(19) United States
(12) Patent Application Publication
(10) Pub. No.: US 2013/0059746 A1
(43) Pub. Date: Mar. 7, 2013

(54) GENE EXPRESSION PROFILING OF CYTOGENETIC ABNORMALITIES

(71) Applicants: John D. Shaughnessy, JR., Roland, AR (US); Yiming Shou, Brookline, MA (US); Qing Zhang, Little Rock, AR (US); Bart Barlogie, Little Rock, AR (US)

(72) Inventors: John D. Shaughnessy, JR., Roland, AR (US); Yiming Shou, Brookline, MA (US); Qing Zhang, Little Rock, AR (US); Bart Barlogie, Little Rock, AR (US)

(73) Assignee: Myeloma Health LLC, New York, NY (US)

(21) Appl. No.: 13/524,589

(22) Filed: Jun. 15, 2012

Related U.S. Application Data
(60) Provisional application No. 61/520,793, filed on Jun. 15, 2011.

(51) Int. Cl. C40B 30/04; C12Q 1/68

(52) U.S. Cl. 506/9; 435/6.14; 435/6.11; 435/6.12

(57) ABSTRACT

Provided herein are methods of predicting cytogenetic abnormalities associated with a cancer in a subject, for example, multiple myeloma. A cytogenetic abnormalities model of a set of reference values obtained from an average of gene expression profile values based on copy number-sensitive genes that correlate to cytogenetic abnormalities associated with the cancer is utilized as a predictive tool. The cytogenetic abnormalities model, as a virtual model (i.e. a “virtual karyotype”), may be tangibly stored with program instructions to implement the model in a computer system. In particular embodiments, the methods and systems provided by the invention operate without FISH (fluorescent in situ hybridization).

### RELATED APPLICATION(S)

This application claims the benefit of U.S. Provisional Application No. 61/520,793, filed on Jun. 15, 2011.

The entire teachings of the above application are incorporated by reference.

### GOVERNMENT SUPPORT

This invention was made with government support under grant CA055819 awarded by the National Cancer Institute. The government has certain rights in the invention.

### FIELD OF THE INVENTION

The present invention generally relates to the field of cancer research. More specifically, the present invention relates to the gene expression profiling of cytogenetic abnormalities.

### BACKGROUND OF THE INVENTION

Multiple myeloma (MM) is an invariantly fatal tumor of terminally differentiated plasma cells (PCs) that home to and expand in the bone marrow. Monoclonal gammopathy of undetermined significance (MGUS) and multiple myeloma are the most frequent forms of monoclonal gammopathies. Monoclonal gammopathy of undetermined significance is the most common plasma cell dyspraxia with an incidence of up to 10% of population over age 75. The molecular basis of monoclonal gammopathy of undetermined significance and multiple myeloma are not very well understood and it is not easy to differentiate these two disorders. Diagnosis of multiple myeloma or monoclonal gammopathy of undetermined significance is identical in ⅔ of cases using classification systems that are based on a combination of clinical criteria such as the amount of bone marrow plasmocytosis, the concentration of monoclonal immunoglobulin in urine or serum, and the presence of bone lesions. Especially in early phases of multiple myeloma, differential diagnosis is associated with a certain degree of uncertainty.

Furthermore, in the diagnosis of multiple myeloma, the clinician must exclude other disorders in which a plasma cell reaction may occur. These other disorders include rheumatoid arthritis, connective tissue disorders, and metastatic carcinoma where the patient may have osteolytic lesions associated with bone metastases. Therefore, given that multiple myeloma is thought to have an extended latency and clinical features are recognized many years after development of the malignancy, new molecular diagnostic techniques are needed for differential diagnosis of multiple myeloma, e.g., monoclonal gammopathy of undetermined significance versus multiple myeloma, or recognition of various subtypes of multiple myeloma.

Multiple myeloma initially resides in the bone marrow, but typically transform into an aggressive disease with increased proliferation (resulting in a higher frequency of abnormal metaphase karyotypes), elevated lactate dehydrogenase (LDH) and extramedullary manifestations (Barlogie B. et al., 2001). Although aneuploidy is observed in more than 90% of cases, cytogenetic abnormalities in this typically hypoproliferative tumor are informative in only about 30% of cases and are typically complex, involving on average seven different chromosomes.

Given this genetic chaos, it has been difficult to establish correlations between genetic abnormalities and clinical outcomes. Only recently has chromosome 13 deletion been identified as a distinct clinical entity with a grave prognosis. However, even with the most comprehensive analysis of laboratory parameters, such as b2-microglobulin (b2M), C-reactive protein (CRP), plasma cell labeling index (PCLI), metaphase karyotyping, and fluorescence in situ hybridization (FISH), the clinical course of patients afflicted with multiple myeloma can only be approximated, because no more than 20% of the clinical heterogeneity can be accounted for. Thus, there are distinct clinical subgroups of multiple myeloma and modern molecular tests may identify these entities. Overall, the progress in understanding the biology and genetics of multiple myeloma has been slow.

The prior art is deficient in correlating gene expression profiling methods to determining cytogenetic abnormalities in a subject, including methods that do not rely on fluorescent in situ hybridization (FISH), which is the current standard in the art for detecting chromosomal abnormalities. The present invention fulfills this need in the art.

### SUMMARY OF THE INVENTION

The present invention provides, inter alia, methods and systems for predicting cytogenetic abnormalities (e.g., chromosomal abnormalities) associated with a cancer in a subject. These methods and systems substitute for FISH (fluorescent in situ hybridization), which is the current standard technique in the art for detecting chromosomal abnormalities. Therefore, while in some embodiments the methods provided by the invention may further provide for detecting a chromosomal abnormality by FISH (e.g. by initial diagnosis before confirmation and/or further testing by the methods and systems provided by the invention or by follow-on testing, following testing by the methods and systems provided by the invention), in certain embodiments, the methods and systems provided by the invention are performed or used without FISH. In a preferred embodiment, the methods and systems provided by the invention are performed or used without FISH.

The methods provided by the invention comprise, in certain embodiments, importing gene expression values obtained from a global gene expression profile of mRNA from cells associated with the cancer into a cytogenetic abnormalities model and predicting, with the model, genes expressing cytogenetic abnormalities in the subject.

The present invention also provides methods for predicting cytogenetic abnormalities in a subject having or at risk for multiple myeloma. The method comprises importing gene expression values obtained from a global gene expression profile of mRNA from plasma cells obtained from the subject into a cytogenetic abnormalities model of a set of reference values of copy number-sensitive genes that correlate to cytogenetic abnormalities associated with multiple myeloma. Using the reference model, genes exhibiting cytogenetic abnormalities in the subject are predicted.

The present invention further provides methods for predicting cytogenetic abnormalities in a subject having or at risk for multiple myeloma. The methods comprise performing global gene expression profiling on mRNA extracted from plasma cells from the subject. Gene expression values obtained from the profile based on copy number-sensitive genes are averaged to reference values correlating to cytogenetic abnormalities associated with (the cancer found in) multiple myeloma. The correlative values of cytogenetic abnormalities comprise a cytogenetic abnormalities model and, thereby, cytogenetic abnormalities in the subject are predicted.

The present invention further still provides computer-readable media tangibly (e.g., non-transiently) storing a virtual model of cytogenetic abnormalities associated with multiple myeloma and implementable in a computer system having a memory, a processor and at least one network connection. The virtual model comprises a list of genes shown in Table 1 identified from global expression profiling of plasma cell mRNA obtained from control multiple myeloma patients, a set of reference values in Table 2 that are averages of the expression values based on copy number-sensitive genes that correlate to cytogenetic abnormalities associated with multiple myeloma; a statistical function to average the gene expression values. The computer-readable medium also tangibly stores program instructions to implement the virtual model in the computer system.

The present invention further still provides methods for predicting cytogenetic abnormalities in a subject having multiple myeloma. The method comprises applying the virtual cytogenetic abnormalities model, comprising the list of genes in Table 1, the reference values in Table 2, the statistical averaging function, and the program instructions of the computer readable medium as described supra in a computer system to average the gene expression values obtained from global expression profiling of mRNA from plasma cells of a subject having multiple myeloma to reference values correlating to cytogenetic abnormalities in multiple myeloma, thereby predicting cytogenetic abnormalities in the subject.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1D depict the distribution of FISH signals in specific chromosome regions: (FIG. 1A) chr1q21, (FIG. 1B) chr1p13, (FIG. 1C) chr13s31, and (FIG. 1D) chr13s285.

### DETAILED DESCRIPTION OF THE INVENTION

A description of example embodiments of the invention follows.

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

Threshold values “substantially similar” to those in Table 2 are within 20, 15, 10, 9, 8, 7, 6, 5, 4, 3, 2, or 1%—in either direction—of the values in Table 2.

“GEP-17,” “GEP-70,” and “GEP-80” are gene expression profiles that are diagnostic and/or prognostic of multiple myeloma and are described more fully in, for example, U.S. Patent Application Publication No. US 2008/0187930, which is incorporated by reference in its entirety, including Table 1 (which provides the GEP-70 signature) and Table 7 (which provides the GEP-17 signature) as well as U.S. Patent Application Publication No. US 2012/0015906, which is incorporated by reference in its entirety, including Table 2. These gene expression profiles may, in certain embodiments, be used in the methods provided by the invention to further characterize a subject, e.g., by diagnosing or further prognosing the subject, in addition to the virtual karyotyping provided by the invention. Additional gene expression profiles for use in this way in the methods provided by the invention include, for example, the 15 gene signature described in U.S. Pat. No. 7,371,736, which is incorporated by reference in its entirety, including Example 12, which describes the 15 gene signature in greater detail.

As used herein, the terms “subject”, “individual” or “patient” refers to a mammal, preferably a human, who has, is suspected of having or at risk for having a pathophysiological condition, for example, but not limited to, multiple myeloma.

As noted above, the invention provides methods and systems for detecting, e.g., chromosomal abnormalities—without FISH, the current state of the art—by virtual karyotyping. These methods and systems utilize the gene expression levels of a set of the copy number sensitive genes of Table 1 located in a chromosomal region suspected of containing a cytogenetic abnormality selected from a gain of chr1q, chr3, chr5, chr7, chr9, chr11, chr15, chr19, or chr21; amplification of chr1q21; or loss of chr1p, chr6q, or chr13q. Thus, for example, to detect a gain of chr1q, a set of the genes listed in Table 1 that are located in region 1q are tested and/or evaluated for their gene expression levels in accordance with the methods provided by the invention. In particular embodiments, at least 10, 20, 30, 40, 50, 60, 70, 80, 90, or 95% of the genes in Table 1 for a given chromosomal region suspected of containing a cytogenetic abnormality are tested and/or evaluated. In other particular embodiments, the expression level of all of the genes in Table 1 for a given chromosomal region suspected of containing a cytogenetic abnormality are tested and/or evaluated.

In other embodiments, expression level of one or more of the genes in Table 9 for a given chromosomal region suspected of containing a cytogenetic abnormality are tested and/or evaluated. Table 9 is a subset of the genes in Table 1, more specifically, the top 10 copy number sensitive genes for the indicated region, ranked according to the correlation between gene expression levels and aCGH. In more particular embodiments, the expression level of at least 2, 3, 4, 5, 6, 7, 8, 9, or all 10 of the genes in Table 9 for a given chromosomal region are tested and/or evaluated. In other particular embodiments, the expression level of the top (by rank of the correlation coefficient in Table 9) 1, 2, 3, 4, or 5 genes in Table 9 for a given chromosomal region are tested and/or evaluated, e.g., the expression level of the top 1 or 2 genes in Table 9 for a given chromosomal region are tested and/or evaluated.

Of course, the methods provided by the invention allow for simultaneous testing for multiple cytogenetic abnormalities in parallel, e.g., one or more cytogenetic abnormalities selected from a gain of chr1q, chr3, chr5, chr7, chr9, chr11, chr15, chr19, or chr21; amplification of chr1q21; or loss of chr1p, chr6q, or chr13q—e.g., the subject can be assayed for the presence of 1, 2, 3, 4, 5, 6, 7, 8, 9, 10, 11, 12, or all 13 cytogenetic abnormalities in parallel. In certain embodiments, the uneven chromosomes are evaluated for the presence of cytogenetic abnormalities by the methods provided by the invention in parallel. In other embodiments, chr1p, chr1q, and chr6q are evaluated for the presence of cytogenetic abnormalities according to the methods provided by the invention in parallel. In still other embodiments, the uneven chromosomes and chr1p, chr1q, and chr6q are evaluated for the presence of cytogenetic abnormalities according to the methods provided by the invention in parallel.

In one embodiment of the present invention there is provided a method for predicting cytogenetic abnormalities associated with a cancer in a subject, comprising importing gene expression values obtained from a global gene expression profile of mRNA from cells associated with the cancer into a cytogenetic abnormalities model; and predicting, with the model, genes expressing cytogenetic abnormalities in the subject.

In this embodiment, the predicting step may comprise averaging the imported gene expression values based on copy number-sensitive genes to reference values correlating to cytogenetic abnormalities associated with the cancer. Further in this embodiment, the cytogenetic abnormalities model may be a virtual model tangibly stored on a computer-readable medium.

In one aspect of this embodiment, the cancer is multiple myeloma and the cytogenetic abnormalities model comprises a set of copy-numbers sensitive genes reference values correlating to cytogenetic abnormalities in Table 2. Particularly, in this aspect, the set of copy number-sensitive genes comprise the genes in Table 1. Furthermore, the reference values may distinguish among DNA amplification, DNA deletion and DNA with normal copy number.

In another embodiment of the present invention, there is provided a method for predicting cytogenetic abnormalities in a subject having or at risk for multiple myeloma, comprising importing gene expression values obtained from a global gene expression profile of mRNA from plasma cells obtained from the subject into a cytogenetic abnormalities model of a set of reference values of copy-numbers sensitive genes correlating to cytogenetic abnormalities associated with multiple myeloma; and predicting, with the reference model, genes exhibiting cytogenetic abnormalities in the subject.

In this embodiment, the copy number-sensitive genes comprise the genes in Table 1. Also, the reference values may comprise the values in Table 2. In addition, the cytogenetic abnormalities predicted by the model may be determinative of a prognosis of the subject having multiple myeloma or may be diagnostic of multiple myeloma in the subject. Furthermore, the reference values and the DNA amplification, deletion or normality represented by the same and the virtual cytogenetic abnormalities model are as described supra.

In yet another embodiment of the present invention, there is provided a method for predicting cytogenetic abnormalities in a subject having or at risk for multiple myeloma, comprising obtaining plasma cells from the subject; performing global gene expression profiling on mRNA extracted from the cells; averaging the gene expression values obtained from the profile based on copy number-sensitive genes to reference values correlating to cytogenetic abnormalities associated with (the cancer found in) multiple myeloma, said correlative values of cytogenetic abnormalities comprising a cytogenetic abnormalities model, thereby predicting cytogenetic abnormalities in the subject.

In this embodiment the copy number-sensitive genes in Table 1, the prognosis and/or diagnosis of multiple myeloma by the cytogenetic abnormalities model, the reference values in Table 2 and the DNA amplification, deletion or normality represented by the same and the virtual reference model are as described supra.

In yet another embodiment of the present invention, there is provided a computer-readable medium tangibly storing a virtual model of cytogenetic abnormalities associated with multiple myeloma and implementable in a computer system having a memory, a processor and at least one network connection, said virtual model comprising a list of genes shown in Table 1 identified from global expression profiling of plasma cell mRNA obtained from control multiple myeloma patients; a set of reference values in Table 2 that are averages of the expression values based on copy number-sensitive genes that correlate to cytogenetic abnormalities associated with multiple myeloma; a statistical function to average the gene expression values; and program instructions to implement the virtual model in the computer system.

In this embodiment, the program instructions may be adapted to receive inputted gene expression values obtained from global expression profiling of mRNA from plasma cells of a subject having multiple myeloma; average the received gene expression values based on copy numbers sensitive genes; and output a value predictive of cytogenetic abnormalities in the subject.

In yet another embodiment of the present invention there is provided a method for predicting cytogenetic abnormalities in a subject having multiple myeloma, comprising applying the virtual model and program instructions of the computer readable medium of claim 21 in a computer system to average the gene expression values obtained from global expression profiling of mRNA from plasma cells of a subject having multiple myeloma to reference values correlating to cytogenetic abnormalities in multiple myeloma, thereby predicting cytogenetic abnormalities in the subject.

Multiple myeloma, a neoplasm of plasma cells, is characterized by complex chromosomal abnormalities, including structural and numerical rearrangements. The cytogenetic abnormalities that are a hallmark of multiple myeloma and other cancers are commonly used as clinical parameters for determining disease stage and guiding therapy decisions for patients. Traditional cytogenetic techniques, including fluorescence in situ hybridization (FISH) and karyotyping, and the recently developed array-based comparative genomic hybridization (aCGH), are widely used to detect chromosomal aberrations and gene copy-number changes. These methods, however, are expensive or time-consuming, or both.

Thus, the present invention provides a virtual cytogenetic abnormalities (vCA) model or cytogenetic abnormalities reference model that uses gene expression profiling to predict cytogenetic abnormalities. The model has accuracy up to about 0.99. The rationale for the model is that disease-associated alterations of genomic regions should in some way alter (“drive”) expression levels of target genes within the regions or nearby; otherwise, the genomic alterations would be just “passengers” without a real contribution to the disease. Therefore, the driving alterations should be predictable via the alteration of expression levels of the genomic region's target genes. Thus, global gene expression profiling can be a one-stop data source for information on molecular diagnosis and/or prognosis, particularly yielding information from the level of specific genes to whole chromosomes for making a molecular diagnosis and/or determination of prognosis in multiple myeloma, as well as potentially other malignancies. Proper analysis of gene expression profiling data can reveal all the information provided by conventional cytogenetic techniques.

The reference model of cytogenetic abnormalities may be a virtual model provided in a computer comprising a computer system or other electronic device having one or more wired or wireless network connections, a memory to store the model and a processor to execute instructions enabling the reference model on the computer or other electronic device. Such computers and electronic devices are well-known and standard in the art. A computer storage medium may tangibly store the virtual reference model and instructions to implement the virtual model in the computer system. As such, the virtual reference model and instructions may comprise a computer program product tangibly stored in a memory on a computer or other computer storage device as are known in the art.

Particularly the virtual cytogenetic abnormalities model may comprise a list of genes identified from global gene expression profiling of mRNA obtained from a biological sample, for example, from plasma cells (e.g. CD138-enriched plasma cells) in the case of multiple myeloma, obtained from a control subjects having the cancer of interest. For example Table 1 provides a list of genes from a subject having multiple myeloma. The model also comprises a set of reference values that are averages of the expression values based on copy number-sensitive genes obtained from global expression profiling of the biological sample that correlate to cytogenetic abnormalities associated with the cancer. For example Table 2 provides these correlative values derived from Table 1. The virtual model also may comprise a statistical function, such as a function to average gene expression values inputted into the model, and the program instructions to implement the virtual model in the computer system.

While the examples provided herein utilize multiple myeloma cells, one of ordinary skill in the art can see that the methods and reference models provided herein are readily adapted to any pathophysiological condition associated with cytogenetic abnormalities during progression and/or remission of the condition. Global gene expression profiling (GEP), whole transcriptome shotgun sequencing (RNA-seq), fluorescent in situ hybridization (FISH), DNA isolation and array-based comparative genomic hybridization (aCGH) or high-throughput DNA sequencing, combining with the statistical analysis techniques provided herein are well-suited to identify copy number-sensitive genes that are associated with a pathophysiological condition, such as, but not limited to a cancer. For example, the reference model described herein can be configured for any cancer.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Study Subjects

Bone marrow aspirates were obtained from patients newly diagnosed with multiple myeloma, who were subsequently treated on NIH-sponsored clinical trials. Patients provided samples under Institutional Review Board—approved informed consent, and records are kept on file. Myeloma plasma cells were isolated from heparinized bone marrow aspirates with an autoMACS device (Miltenyi Biotec, Inc., Auburn, Calif.) using CD138-based immunomagnetic bead selection, as previously described (Zhan, 2002).

DNA Isolation and Array-Based Comparative Genomic Hybridization (aCGH)

High-molecular-weight genomic DNA was isolated from aliquots of CD138-enriched plasma cells with the use of the QIAamp DNA mini kit (Qiagen, Valencia, Calif.). Tumor- and sex-matched reference genomic DNA (Promega Corp., Madison, Wis.) was hybridized to the Agilent 244K aCGH array according to the manufacturer's instructions (Agilent Technologies, Inc., Santa Clara, Calif.).

### Interphase Fluorescence In Situ Hybridization

Bone marrow aspirates from patients with multiple myeloma were processed to remove erythrocytes. Copy-number changes in myeloma plasma cells were detected by triple-color interphase FISH analysis of chromosome loci, as described (Shaughnessy, 2000). Bacterial artificial chromosome (BAC) clones specific for 1q21 (CKS1B), 1p13 (AHCYL1), 13q14 (D13S31), and 13q34 (D13S285) were obtained from BACPAC Resources Center (Oakland, Calif.) and labeled with Spectrum Red- or Spectrum Green-conjugated nucleotides via nick translation (Vysis, Downers Grove, Ill.). At least 100 myeloma cells stained with immunoglobulin (Ig) light-chain antibody (kappa or lambda) conjugated with 7-amino-4-methylcoumarin-3-acetic acid (AMCA) were counted for copies of each probe. The threshold of significant abnormality (gain or loss) of each probe was set at ≧20%, as previously described (Shaughnessy et al. Blood, 15 Aug. 2000).

### Cytogenetics

Bone marrow was processed for chromosome studies by standard techniques. A direct harvest, a 24-hour unsynchronized culture, and a 48-hour synchronized culture were employed on most specimens. The 24-hour culture employed the adding of ethidium bromide (10 μg/mL) to the culture 2 hours prior to harvest, with an additional 1 hour in Colcemid solution (0.05 μg/mL). The 48-hour synchronized cultures employed a 17-hour exposure of cells to 10-7 M methotrexate. Cells were washed with unsupplemented medium and then released with 10-5 M thymidine. Colcemid (0.05 μg/mL) was added 5 hours later for 1 hour. For the purpose of cytogenetic examination, an effort was made to examine at least 20 metaphases, with the application of Giemsa banding techniques. The presence of cytogenetic abnormalities required the detection of at least two abnormal metaphases in cases of hyperdiploidy and translocations, whereas at least three metaphases with clonal abnormalities were required in cases of whole and partial chromosome deletions.

### RNA Purification and Microarray Hybridization

RNA purification, cDNA synthesis, cRNA preparation, and hybridization to the Human Genome U133Plus 2.0 GeneChip microarray (Affymetrix, Santa Clara, Calif.) were performed as previously described (Zhan, 2006; Shaughnessy, 2007; Zhan, 2007).

### Data Analyses

A modified Lowess algorithm was used to normalize aCGH data (Yang, 2002). Statistically, altered regions were identified with the use of a circular binary segmentation algorithm (Yang, 2002). The MASS algorithm was used to summarize and normalize Affymetrix U133Plus2.0 expression data. All statistical analyses were performed with the statistics software R (version 2.6.2; available free of charge at www.r-project.org) and R packages developed by the BioConductor project (available free of charge at www.bioconductor.org).

DNA copy number-sensitive genes were determined by the following procedures. First, Pearson's correlation coefficient (PCC) of gene expression levels and the copy numbers of the corresponding DNA loci were calculated. Second, the column labels of both gene expression levels and the DNA loci copy numbers were permuted, and the random correlation coefficients were calculated for each gene based on the permuted matrices. Third, the cutoff value of Pearson's correlation coefficient was then determined at 0.35 so that the false-discovery rate (FDR) was <0.05, as only 56 genes had random correlation coefficients >0.35 instead of 1,114 genes based the original matrix (FDR=56/1114). The other gene expression data of newly diagnosed MM samples can be downloaded from National Center for Biotechnology Information (NCBI) Gene Expression Omnibus (GEO) Website (www.ncbi.nlm.nih.gov/geo/); the accession number for the data sets is GSE2658 (Shaughnessy, 2007).

### Example 2

### Determination of Copy Numbers Sensitive Genes

Genome-wide gene expression profiles and DNA copy numbers (CNs) in purified plasma cell samples obtained from 92 newly diagnosed MM patients, using the Affymetrix GeneChip and the Agilent aCGH platforms, respectively. DNA copy number-sensitive genes were determined by Pearson's correlation coefficient (PCC) of gene expression levels and the copy numbers of the corresponding DNA loci. Applying the criterion of PCC >0.35, which kept the false-discovery rate to <5%, 1,114 copy numbers-sensitive genes were identified (Table 1).

On the basis of these copy number-sensitive genes, a vCA model was developed for predicting cytogenetic abnormalities in multiple myeloma patients by means of gene expression profiling. The model focuses particularly on chromosomes 3, 5, 7, 9, 11, 13, 15, 19, and 21, as well as the 1p, 1q, and 6q segments, which are the most commonly altered chromosome regions in myeloma plasma cells.

TABLE 1
Genes in the vCA Model and their location

| Symbol | Location | Symbol | Location | AMPD1 | chr1p |
|---|---|---|---|---|---|
| AASDHPPT | chr11 | AMPD1 | chr1p | AMPD2 | chr1p |
| ABHD13 | chr13 | AMPD2 | chr1p | AMPH | chr7 |
| ABHD2 | chr15 | AMPH | chr7 | ANGEL1 | chr14 |
| ACO1 | chr9 | ANGEL1 | chr14 | ANKRD10 | chr13 |
| ACPL2 | chr3 | ANKRD10 | chr13 | ANKRD11 | chr16 |
| ACSL5 | chr10 | ANKRD11 | chr16 | ANKRD12 | chr18 |
| ADAM10 | chr15 | ANKRD12 | chr18 | ANKRD13C | chr1p |
| ADAM19 | chr5 | ANKRD13C | chr1p | ANKRD15 | chr9 |
| ADAMTSL4 | chr1q/chr1q21 | ANKRD15 | chr9 | ANKRD45 | chr1q |
| ADCK2 | chr7 | ANKRD45 | chr1q | ANKRD49 | chr11 |
| ADCY7 | chr16 | ANKRD49 | chr11 | ANP32E | chr1q/chr1q21 |
| ADRB2 | chr5 | ANP32E | chr1q/chr1q21 | AP1G1 | chr16 |
| AGL | chr1p | AP1G1 | chr16 | AP3S2 | chr15 |
| AGPAT3 | chr21 | AP3S2 | chr15 | AP4B1 | chr1p |
| AHCYL1 | chr1p | AP4B1 | chr1p | AP4S1 | chr14 |
| AHI1 | chr6q/chr6 | AP4S1 | chr14 | APC | chr5 |
| AIG1 | chr6q/chr6 | APC | chr5 | APEX1 | chr14 |
| AK3 | chr9 | APEX1 | chr14 | APH1A | chr1q/chr1q21 |
| AKAP11 | chr13 | APH1A | chr1q/chr1q21 | APTX | chr9 |
| ALDH9A1 | chr1q | APTX | chr9 | ARHGAP1 | chr11 |
| ALG5 | chr13 | ARHGAP1 | chr11 | ARHGAP11A | chr15 |
| ALKBH3 | chr11 | ARHGAP11A | chr15 | ARHGAP30 | chr1q |
| ALOX5AP | chr13 | ARHGAP30 | chr1q | ARHGAP5 | chr14 |
| AMD1 | chr6q/chr6 | ARHGAP5 | chr14 | AMPD1 | chr1p |
| AURKC | chr19 | C11orf57 | chr11 | C16orf57 | chr16 |
| AVEN | chr15 | C11orf73 | chr11 | C16orf61 | chr16 |
| B3GALTL | chr13 | C12orf23 | chr12 | C16orf80 | chr16 |
| BAG1 | chr9 | C12orf31 | chr12 | C17orf81 | chr17 |
| BAG5 | chr14 | C13orf1 | chr13 | C17orf85 | chr17 |
| BAIAP2L1 | chr7 | C13orf23 | chr13 | C18orf19 | chr18 |
| BCAS2 | chr1p | C13orf34 | chr13 | C18orf21 | chr18 |
| BCL10 | chr1p | C13orf7 | chr13 | C18orf37 | chr18 |
| BFSP2 | chr3 | C13orf8 | chr13 | C19orf26 | chr19 |
| BIN3 | chr8 | C14orf102 | chr14 | C1orf106 | chr1q |
| BIRC2 | chr11 | C14orf108 | chr14 | C1orf107 | chr1q |
| BIRC3 | chr11 | C14orf122 | chr14 | C1orf112 | chr1q |
| BLCAP | chr20 | C14orf124 | chr14 | C1orf156 | chr1q |
| BNIP1 | chr5 | C14orf133 | chr14 | C1orf19 | chr1q |
| BOLA1 | chr1q/chr1q21 | C14orf149 | chr14 | C1orf2 | chr1q |
| BOP1 | chr8 | C14orf153 | chr14 | C1orf21 | chr1q |
| BRD7 | chr16 | C14orf156 | chr14 | C1orf25 | chr1q |
| BRMS1 | chr11 | C14orf166 | chr14 | C1orf52 | chr1p |
| BRP44 | chr1q | C14orf2 | chr14 | C1orf56 | chr1q/chr1q21 |
| BRP44L | chr6q/chr6 | C14orf28 | chr14 | C1orf74 | chr1q |
| BRWD1 | chr21 | C14orf4 | chr14 | C1orf85 | chr1q |
| BXDC1 | chr6q/chr6 | C15orf17 | chr15 | C20orf11 | chr20 |
| BXDC5 | chr1p | C15orf29 | chr15 | C20orf121 | chr20 |
| C11orf2 | chr11 | C15orf40 | chr15 | C20orf29 | chr20 |
| C20orf77 | chr20 | C9orf30 | chr9 | CDC37L1 | chr9 |
| C21orf33 | chr21 | C9orf82 | chr9 | CDC42BPA | chr1q |
| C3orf17 | chr3 | CA12 | chr15 | CDC42BPB | chr14 |
| C3orf28 | chr3 | CACYBP | chr1q | CDC42EP3 | chr2 |
| C3orf31 | chr3 | CASP4 | chr11 | CDC42SE1 | chr1q/chr1q21 |
| C3orf33 | chr3 | CASP8AP2 | chr6q/chr6 | CDC73 | chr1q |
| C4orf15 | chr4 | CBFB | chr16 | CDCA4 | chr14 |
| C5orf24 | chr5 | CCBL1 | chr9 | CDKN1B | chr12 |
| C5orf5 | chr5 | CCDC126 | chr7 | CDS2 | chr20 |
| C6orf113 | chr6q/chr6 | CCDC25 | chr8 | CEACAM6 | chr19 |
| C6orf120 | chr6q/chr6 | CCDC28A | chr6q/chr6 | CENPJ | chr13 |
| C6orf130 | chr6 | CCDC52 | chr3 | CENPL | chr1q |
| C6orf136 | chr6 | CCDC82 | chr11 | CENPT | chr16 |
| C6orf151 | chr6 | CCDC90B | chr11 | CENTD2 | chr11 |
| C6orf66 | chr6q/chr6 | CCNC | chr6q/chr6 | CEP164 | chr11 |
| C6orf70 | chr6q/chr6 | CCND1 | chr11 | CEP170 | chr1q |
| C7orf23 | chr7 | CCNE1 | chr19 | CEP192 | chr18 |
| C7orf41 | chr7 | CCNK | chr14 | CEP27 | chr15 |
| C7orf46 | chr7 | CCT3 | chr1q | CEP57 | chr11 |
| C8orf41 | chr8 | CD164 | chr6q/chr6 | CEP76 | chr18 |
| C8orf58 | chr8 | CD48 | chr1q | CEPT1 | chr1p |
| C9orf103 | chr9 | CD55 | chr1q | CES2 | chr16 |
| C9orf23 | chr9 | CDC16 | chr13 | CFDP1 | chr16 |
| C9orf25 | chr9 | CDC2L6 | chr6q/chr6 | CG018 | chr13 |
| CGRRF1 | chr14 | CNOT1 | chr16 | CTSK | chr1q/chr1q21 |
| CHD1L | chr1q/chr1q21 | CNOT7 | chr8 | CTSZ | chr20 |
| CHD6 | chr20 | CNTNAP3 | chr9 | CUL4A | chr13 |
| CHD8 | chr14 | COG2 | chr1q | CUL5 | chr11 |
| CHD9 | chr16 | COG3 | chr13 | CWF19L2 | chr11 |
| CHMP4A | chr14 | COG6 | chr13 | CYB5B | chr16 |
| CHMP7 | chr8 | COMMD6 | chr13 | CYBASC3 | chr11 |
| CHODL | chr21 | COPS2 | chr15 | CYC1 | chr8 |
| CHRAC1 | chr8 | COQ9 | chr16 | CYLD | chr16 |
| CHRNA5 | chr15 | COX4I1 | chr16 | CYP3A5 | chr7 |
| CHURC1 | chr14 | COX4NB | chr16 | DAB2 | chr5 |
| CIAPIN1 | chr16 | COX7A2 | chr6q/chr6 | DARS2 | chr1q |
| CIB2 | chr15 | COX7C | chr5 | DBNDD2 | chr20 |
| CILP | chr15 | CREB3L4 | chr1q/chr1q21 | DBT | chr1p |
| CIRH1A | chr16 | CREBL2 | chr12 | DCP2 | chr5 |
| CITED2 | chr6q/chr6 | CRYL1 | chr13 | DCTN3 | chr9 |
| CKLF | chr16 | CSDE1 | chr1p | DCTN5 | chr16 |
| CKS1B | chr1q/chr1q21 | CSE1L | chr20 | DCUN1D5 | chr11 |
| CLCC1 | chr1p | CSNK1G1 | chr15 | DDR2 | chr1q |
| CLK2 | chr1q | CSNK1G3 | chr5 | DDX10 | chr11 |
| CLK4 | chr5 | CSTF1 | chr20 | DDX19A | chr16 |
| CLN5 | chr13 | CSTF3 | chr11 | DDX20 | chr1p |
| CLNS1A | chr11 | CTBS | chr1p | DDX24 | chr14 |
| CLTA | chr9 | CTDP1 | chr18 | DDX28 | chr16 |
| DDX58 | chr9 | DSCR3 | chr21 | ELK4 | chr1q |
| DDX59 | chr1q | DUSP12 | chr1q | ELL2 | chr5 |
| DEDD | chr1q | DUSP23 | chr1q | ELMO1 | chr7 |
| DENND1C | chr19 | DYM | chr18 | ELMO2 | chr20 |
| DENND2C | chr1p | DYNLT1 | chr6q/chr6 | ELOVL7 | chr5 |
| DENND4A | chr15 | E2F3 | chr6 | ELP3 | chr8 |
| DET1 | chr15 | EBPL | chr13 | ENSA | chr1q/chr1q21 |
| DHRS1 | chr14 | ECHDC1 | chr6q/chr6 | ENY2 | chr8 |
| DHX29 | chr5 | EDC3 | chr15 | EPB41L4A | chr5 |
| DIDO1 | chr20 | EDC4 | chr16 | EPHB1 | chr3 |
| DLST | chr14 | EDEM3 | chr1q | EPSTI1 | chr13 |
| DMPK | chr19 | EDG3 | chr9 | ERCC5 | chr13 |
| DNAH1 | chr3 | EEF1E1 | chr6 | ERCC8 | chr5 |
| DNAJC15 | chr13 | EFHA1 | chr13 | ERH | chr14 |
| DNAJC18 | chr5 | EFNA4 | chr1q | ERICH1 | chr8 |
| DNTTIP2 | chr1p | EFTUD1 | chr15 | ESCO1 | chr18 |
| DOCK8 | chr9 | EGFR | chr7 | ESD | chr13 |
| DOCK9 | chr13 | EID1 | chr15 | ESRRA | chr11 |
| DPF2 | chr11 | EIF2B2 | chr14 | ETFA | chr15 |
| DPH5 | chr1p | EIF2S1 | chr14 | EVI5 | chr1p |
| DPM1 | chr20 | ELAC1 | chr18 | EVL | chr14 |
| DPM3 | chr1q | ELAVL1 | chr19 | EXT2 | chr11 |
| DPP3 | chr11 | ELF1 | chr13 | F2R | chr5 |
| DR1 | chr1p | ELF5 | chr11 | FAM103A1 | chr15 |
| FAM20B | chr1q | FGFR1OP | chr6q/chr6 | GNG5 | chr1p |
| FAM44B | chr5 | FIZ1 | chr19 | GOLGA5 | chr14 |
| FAM46C | chr1p | FLAD1 | chr1q/chr1q21 | GOLGA7 | chr8 |
| FAM48A | chr13 | FLI1 | chr11 | GON4L | chr1q |
| FAM76B | chr11 | FNDC3A | chr13 | GOPC | chr6q/chr6 |
| FAM96B | chr16 | FNTA | chr8 | GPD1L | chr3 |
| FANCD2 | chr3 | FUCA2 | chr6q/chr6 | GPLD1 | chr6 |
| FANCE | chr6 | FXC1 | chr11 | GPR137B | chr1q |
| FANCG | chr9 | GAB2 | chr11 | GPR180 | chr13 |
| FARP2 | chr2 | GALT | chr9 | GTF2B | chr1p |
| FARS2 | chr6 | GAPVD1 | chr9 | GTF2E2 | chr8 |
| FBXL14 | chr12 | GARNL3 | chr9 | GTF2F1 | chr19 |
| FBXL3 | chr13 | GATAD2B | chr1q/chr1q21 | GTF2F2 | chr13 |
| FBXL8 | chr16 | GBA | chr1q | GTF3C4 | chr9 |
| FBXO22 | chr15 | GBA2 | chr9 | GTPBP8 | chr3 |
| FBXO25 | chr8 | GDA | chr9 | GYG1 | chr3 |
| FBXO28 | chr1q | GGPS1 | chr1q | HAPLN4 | chr19 |
| FBXO3 | chr11 | GLG1 | chr16 | HBS1L | chr6q/chr6 |
| FBXO33 | chr14 | GLRX5 | chr14 | HBXIP | chr1p |
| FCHSD2 | chr11 | GMFB | chr14 | HDAC2 | chr6q/chr6 |
| FDFT1 | chr8 | GMPR2 | chr14 | HDAC3 | chr5 |
| FDPS | chr1q | GNAI3 | chr1p | HDDC2 | chr6q/chr6 |
| FEM1B | chr15 | GNB2L1 | chr5 | HDHD2 | chr18 |
| FER | chr5 | GNG11 | chr7 | HEBP2 | chr6q/chr6 |
| HHLA3 | chr1p | IL6R | chr1q/chr1q21 | KBTBD6 | chr13 |
| HIAT1 | chr1p | ILF2 | chr1q/chr1q21 | KBTBD7 | chr13 |
| HIGD2A | chr5 | INTS10 | chr8 | KCNMB3 | chr3 |
| HIPK1 | chr1p | INTS3 | chr1q/chr1q21 | KCTD13 | chr16 |
| HISPPD2A | chr15 | INTS6 | chr13 | KCTD20 | chr6 |
| HMGA1 | chr6 | IQCE | chr7 | KCTD5 | chr16 |
| HOMER1 | chr5 | IQGAP3 | chr1q | KCTD6 | chr3 |
| HOXA5 | chr7 | IQWD1 | chr1q | KIAA0133 | chr1q |
| HS2ST1 | chr1p | IRAK2 | chr3 | KIAA0174 | chr16 |
| HSBP1 | chr16 | ISG20L2 | chr1q | KIAA0182 | chr16 |
| HSPC171 | chr16 | ISL1 | chr5 | KIAA0317 | chr14 |
| HSPH1 | chr13 | ISL2 | chr15 | KIAA0323 | chr14 |
| HUS1 | chr7 | ITCH | chr20 | KIAA0329 | chr14 |
| IARS2 | chr1q | ITFG1 | chr16 | KIAA0406 | chr20 |
| IBTK | chr6q/chr6 | ITPK1 | chr14 | KIAA0423 | chr14 |
| IDH3A | chr15 | IVNS1ABP | chr1q | KIAA0460 | chr1q/chr1q21 |
| IDH3B | chr20 | JAK2 | chr9 | KIAA0513 | chr16 |
| IDUA | chr4 | JARID2 | chr6 | KIAA0652 | chr11 |
| IFNGR2 | chr21 | JMJD1B | chr5 | KIAA0859 | chr1q |
| IFT52 | chr20 | JOSD3 | chr11 | KIAA0999 | chr11 |
| IGF2R | chr6q/chr6 | JRKL | chr11 | KIAA1219 | chr20 |
| IKBKB | chr8 | KATNB1 | chr16 | KIAA1704 | chr13 |
| IL10RB | chr21 | KBTBD2 | chr7 | KIAA1797 | chr9 |
| HHLA3 | chr1p | KBTBD4 | chr11 | KIAA1967 | chr8 |
| KIAA2026 | chr9 | LOC93349 | chr2 | MARK3 | chr14 |
| KIF13B | chr8 | LONRF1 | chr8 | MATR3 | chr5 |
| KIF14 | chr1q | LPXN | chr11 | MAX | chr14 |
| KIF21B | chr1q | LRIG2 | chr1p | MBD1 | chr18 |
| KIFAP3 | chr1q | LRRC57 | chr15 | MBNL2 | chr13 |
| KLC2 | chr11 | LRRC8D | chr1p | MCPH1 | chr8 |
| KLHL18 | chr3 | LSG1 | chr3 | MED19 | chr11 |
| KLHL20 | chr1q | LSM1 | chr8 | MED4 | chr13 |
| KLHL26 | chr19 | LSM11 | chr5 | MED6 | chr14 |
| KPNA1 | chr3 | LSM5 | chr7 | MEIS2 | chr15 |
| KPNA3 | chr13 | LTV1 | chr6q/chr6 | MEN1 | chr11 |
| LACTB | chr15 | LY6E | chr8 | METTL3 | chr14 |
| LAMP1 | chr13 | LY9 | chr1q | METTL4 | chr18 |
| LANCL2 | chr7 | MAB21L1 | chr13 | MGC13379 | chr11 |
| LASS2 | chr1q/chr1q21 | MAFK | chr7 | MGC70857 | chr8 |
| LCMT2 | chr15 | MAK10 | chr9 | MGST3 | chr1q |
| LEAP2 | chr5 | MAN1A2 | chr1p | MIER3 | chr5 |
| LEPROTL1 | chr8 | MANBAL | chr20 | MIZF | chr11 |
| LIG4 | chr13 | MAP1LC3B | chr16 | MKKS | chr20 |
| LIN7C | chr11 | MAP2K4 | chr17 | MNS1 | chr15 |
| LINS1 | chr15 | MAP2K5 | chr15 | MON1B | chr16 |
| LMO4 | chr1p | MAP3K4 | chr6q/chr6 | MPPE1 | chr18 |
| LNX2 | chr13 | MAPBPIP | chr1q | MRE11A | chr11 |
| LOC51035 | chr11 | MARK1 | chr1q | MRLC2 | chr18 |
| MRP63 | chr13 | MX2 | chr21 | NOL3 | chr16 |
| MRPL18 | chr6q/chr6 | MYC | chr8 | NPAT | chr11 |
| MRPL22 | chr5 | MYCBP2 | chr13 | NR1H3 | chr11 |
| MRPL9 | chr1q/chr1q21 | MYH14 | chr19 | NR1I2 | chr3 |
| MRPS14 | chr1q | MYNN | chr3 | NRAS | chr1p |
| MRPS21 | chr1q/chr1q21 | MYST3 | chr8 | NRG2 | chr5 |
| MRPS25 | chr3 | MZF1 | chr19 | NRXN3 | chr14 |
| MRPS27 | chr5 | N4BP1 | chr16 | NSFL1C | chr20 |
| MRPS31 | chr13 | NARG1L | chr13 | NT5DC1 | chr6q/chr6 |
| MRPS36 | chr5 | NARG2 | chr15 | NUDT15 | chr13 |
| MSL2L1 | chr3 | NAT11 | chr11 | NUDT3 | chr6 |
| MSTO1 | chr1q | NDEL1 | chr17 | NUDT4 | chr12 |
| MTA1 | chr14 | NDFIP2 | chr13 | NUF2 | chr1q |
| MTF2 | chr1p | NDUFS2 | chr1q | NUFIP1 | chr13 |
| MTFMT | chr15 | NDUFS4 | chr5 | NUP153 | chr6 |
| MTIF3 | chr13 | NEDD8 | chr14 | NUP160 | chr11 |
| MTMR11 | chr1q/chr1q21 | NEK2 | chr1q | NUP205 | chr7 |
| MTMR4 | chr17 | NES | chr1q | NUP37 | chr12 |
| MTMR9 | chr8 | NFIX | chr19 | NUP43 | chr6q/chr6 |
| MTRF1L | chr6q/chr6 | NIP30 | chr16 | NUP93 | chr16 |
| MTUS1 | chr8 | NIPSNAP3B | chr9 | NUP98 | chr11 |
| MTX1 | chr1q | NISCH | chr3 | NVL | chr1q |
| MUC1 | chr1q | NIT1 | chr1q | ODF2 | chr9 |
| MUTED | chr6 | NNT | chr5 | OGFOD1 | chr16 |
| OGG1 | chr3 | PDCD2 | chr6q/chr6 | PIK3C3 | chr18 |
| OPA3 | chr19 | PDE1C | chr7 | PIP5K1A | chr1q/chr1q21 |
| OPN3 | chr1q | PDE7A | chr8 | PKM2 | chr15 |
| OR7A5 | chr19 | PDE8A | chr15 | PKN2 | chr1p |
| OR7C2 | chr19 | PDPR | chr16 | PLA2G4A | chr1q |
| OSBPL10 | chr3 | PEX16 | chr11 | PLAGL2 | chr20 |
| OSTM1 | chr6q/chr6 | PEX19 | chr1q | PLCG2 | chr16 |
| OXA1L | chr14 | PEX3 | chr6q/chr6 | PMF1 | chr1q |
| OXNAD1 | chr3 | PEX5 | chr12 | PML | chr15 |
| P15RS | chr18 | PEX7 | chr6q/chr6 | PMVK | chr1q/chr1q21 |
| PABPN1 | chr14 | PFDN4 | chr20 | PNMA1 | chr14 |
| PAK1 | chr11 | PHF11 | chr13 | PNOC | chr8 |
| PAN3 | chr13 | PHF14 | chr7 | POGK | chr1q |
| PAPOLA | chr14 | PHF20L1 | chr8 | POGZ | chr1q/chr1q21 |
| PARP16 | chr15 | PHKB | chr16 | POLI | chr18 |
| PASK | chr2 | PIAS2 | chr18 | POLR1B | chr2 |
| PBX1 | chr1q | PIAS3 | chr1q/chr1q21 | POLR1D | chr13 |
| PCBD2 | chr5 | PICALM | chr11 | POLR1E | chr9 |
| PCCA | chr13 | PIGB | chr15 | POLR2C | chr16 |
| PCF11 | chr11 | PIGC | chr1q | POLR3B | chr12 |
| PCID2 | chr13 | PIGH | chr14 | POLR3C | chr1q/chr1q21 |
| PCM1 | chr8 | PIGK | chr1p | POLR3D | chr8 |
| PCMT1 | chr6q/chr6 | PIGM | chr1q | POMP | chr13 |
| PCNT | chr21 | PIGU | chr20 | PPIL4 | chr6q/chr6 |
| PPOX | chr1q | PSME1 | chr14 | RASSF5 | chr1q |
| PPP2CB | chr8 | PSPC1 | chr13 | RBBP8 | chr18 |
| PPP2R1B | chr11 | PTK2B | chr8 | RBL2 | chr16 |
| PPP2R2A | chr8 | PTPN2 | chr18 | RBM13 | chr8 |
| PPP3CC | chr8 | PTTG1IP | chr21 | RBM16 | chr6q/chr6 |
| PRCC | chr1q | PUS3 | chr11 | RBM25 | chr14 |
| PREP | chr6q/chr6 | QKI | chr6q/chr6 | RBM26 | chr13 |
| PRKAA1 | chr5 | QRSL1 | chr6q/chr6 | RBM7 | chr11 |
| PRKAB2 | chr1q/chr1q21 | RAB14 | chr9 | RBM8A | chr1q/chr1q21 |
| PRKACB | chr1p | RAB1B | chr11 | RCBTB1 | chr13 |
| PRKRIR | chr11 | RAB22A | chr20 | RCBTB2 | chr13 |
| PRMT5 | chr14 | RAB3GAP2 | chr1q | RCOR3 | chr1q |
| PRMT6 | chr1p | RAB7L1 | chr1q | RDH11 | chr14 |
| PROSC | chr8 | RAB8B | chr15 | RDX | chr11 |
| PRPF3 | chr1q/chr1q21 | RABIF | chr1q | RELA | chr11 |
| PRR3 | chr6 | RAC1 | chr7 | REPS1 | chr6q/chr6 |
| PRR7 | chr5 | RAD50 | chr5 | REV3L | chr6q/chr6 |
| PRUNE | chr1q/chr1q21 | RAE1 | chr20 | RFWD2 | chr1q |
| PSIP1 | chr9 | RALBP1 | chr18 | RFXAP | chr13 |
| PSMA5 | chr1p | RALGPS1 | chr9 | RFXDC2 | chr15 |
| PSMB1 | chr6q/chr6 | RANBP10 | chr16 | RGMB | chr5 |
| PSMB10 | chr16 | RANBP5 | chr13 | RGS19 | chr20 |
| PSMD4 | chr1q/chr1q21 | RANBP6 | chr9 | RGS5 | chr1q |
| PSMD7 | chr16 | RAPGEF1 | chr9 | RGS7 | chr1q |
| RHOG | chr11 | RPS23 | chr5 | SEMA4D | chr9 |
| RICTOR | chr5 | RPS6 | chr9 | SEP15 | chr1p |
| RIOK1 | chr6 | RRAGA | chr9 | SEP9 | chr17 |
| RIPK5 | chr1q | RSBN1 | chr1p | SETD3 | chr14 |
| RIT1 | chr1q | RSF1 | chr11 | SETD4 | chr21 |
| RLN2 | chr9 | RSRC1 | chr3 | SETDB1 | chr1q/chr1q21 |
| RNASEH2B | chr13 | RWDD1 | chr6q/chr6 | SETDB2 | chr13 |
| RNASET2 | chr6q/chr6 | RWDD3 | chr1p | SF3A2 | chr19 |
| RNF138 | chr18 | S100A10 | chr1q/chr1q21 | SF3B4 | chr1q/chr1q21 |
| RNF14 | chr5 | S100A11 | chr1q/chr1q21 | SFRS5 | chr14 |
| RNF146 | chr6q/chr6 | SAAL1 | chr11 | SFT2D1 | chr6q/chr6 |
| RNF31 | chr14 | SAP18 | chr13 | SFT2D2 | chr1q |
| RNF38 | chr9 | SARS | chr1p | SH2D1B | chr1q |
| RNF6 | chr13 | SAT2 | chr17 | SH3BP5L | chr1q |
| RNF7 | chr3 | SBF2 | chr11 | SH3GLB1 | chr1p |
| RNMT | chr18 | SC5DL | chr11 | SHPRH | chr6q/chr6 |
| RNMTL1 | chr17 | SCAMP5 | chr15 | SIDT1 | chr3 |
| RNPEP | chr1q | SCNM1 | chr1q/chr1q21 | SIKE | chr1p |
| RPL17 | chr18 | SCYL3 | chr1q | SIPA1L1 | chr14 |
| RPL36AL | chr14 | SDHC | chr1q | SKP2 | chr5 |
| RPL37 | chr5 | SEC23A | chr14 | SLC23A1 | chr5 |
| RPLP1 | chr15 | SEC63 | chr6q/chr6 | SLC25A38 | chr3 |
| RPP40 | chr6 | SEH1L | chr18 | SLC25A44 | chr1q |
| RPS12 | chr6q/chr6 | SELL | chr1q | SLC25A45 | chr11 |
| SLC30A7 | chr1p | SOCS4 | chr14 | TAF1C | chr16 |
| SLC35A3 | chr1p | SPATA2 | chr20 | TAF4 | chr20 |
| SLC35B3 | chr6 | SPATA5L1 | chr15 | TAF5L | chr1q |
| SLC35F2 | chr11 | SPG20 | chr13 | TAF6L | chr11 |
| SLC39A14 | chr8 | SPG7 | chr16 | TAGAP | chr6q/chr6 |
| SLC41A3 | chr3 | SPTLC2 | chr14 | TAGLN2 | chr1q |
| SLC7A1 | chr13 | SRD5A1 | chr5 | TARBP1 | chr1q |
| SLC7A6 | chr16 | SS18L1 | chr20 | TATDN2 | chr3 |
| SLC7A6OS | chr16 | SSH2 | chr17 | TBC1D13 | chr9 |
| SMAD2 | chr18 | STK24 | chr13 | TBCC | chr6 |
| SMEK1 | chr14 | STK35 | chr20 | TBCCD1 | chr3 |
| SMPD1 | chr11 | STK38L | chr12 | TBP | chr6q/chr6 |
| SMURF1 | chr7 | STRAP | chr12 | TBPL1 | chr6q/chr6 |
| SNF1LK | chr21 | STX16 | chr20 | TCOF1 | chr5 |
| SNRPB | chr20 | STX6 | chr1q | TCP1 | chr6q/chr6 |
| SNRPD1 | chr18 | STXBP3 | chr1p | TDP1 | chr14 |
| SNUPN | chr15 | SUCLA2 | chr13 | TDRD3 | chr13 |
| SNW1 | chr14 | SUGT1 | chr13 | TERF2 | chr16 |
| SNX11 | chr17 | SUPT16H | chr14 | TERF2IP | chr16 |
| SNX14 | chr6q/chr6 | SV2B | chr15 | TEX10 | chr9 |
| SNX19 | chr11 | SYNCRIP | chr6q/chr6 | TFB1M | chr6q/chr6 |
| SNX27 | chr1q/chr1q21 | SYNJ1 | chr21 | TGDS | chr13 |
| SNX5 | chr20 | TADA1L | chr1q | TH1L | chr20 |
| SNX6 | chr14 | TAF11 | chr6 | THBS3 | chr1q |
| THEM2 | chr6 | TMEM24 | chr11 | TRIM4 | chr7 |
| THEM4 | chr1q/chr1q21 | TMEM55B | chr14 | TRIM48 | chr11 |
| THG1L | chr5 | TMEM77 | chr1p | TRIM58 | chr1q |
| TIMM17A | chr1q | TNFSF10 | chr3 | TRNT1 | chr3 |
| TINF2 | chr14 | TNKS | chr8 | TSC22D1 | chr13 |
| TINP1 | chr5 | TNN | chr1q | TSEN34 | chr19 |
| TIPRL | chr1q | TOMM34 | chr20 | TSPYL1 | chr6q/chr6 |
| TIRAP | chr11 | TP53 | chr17 | TSSC4 | chr11 |
| TM2D3 | chr15 | TP53RK | chr20 | TTBK2 | chr15 |
| TM6SF2 | chr19 | TPM1 | chr15 | TTC1 | chr5 |
| TM9SF2 | chr13 | TPM3 | chr1q/chr1q21 | TTC5 | chr14 |
| TM9SF4 | chr20 | TPP2 | chr13 | TTC9C | chr11 |
| TMCO1 | chr1q | TPR | chr1q | TTLL7 | chr1p |
| TMED5 | chr1p | TRAF3 | chr14 | TUBB4 | chr19 |
| TMEM1 | chr21 | TRAF3IP3 | chr1q | TUBE1 | chr6q/chr6 |
| TMEM107 | chr17 | TRAPPC2L | chr16 | TUBGCP3 | chr13 |
| TMEM108 | chr3 | TRAT1 | chr3 | TULP4 | chr6q/chr6 |
| TMEM123 | chr11 | TRIM13 | chr13 | TWSG1 | chr18 |
| TMEM126A | chr11 | TRIM14 | chr9 | TXNDC1 | chr14 |
| TMEM126B | chr11 | TRIM21 | chr11 | TXNL1 | chr18 |
| TMEM133 | chr11 | TRIM26 | chr6 | TXNL4A | chr18 |
| TMEM135 | chr11 | TRIM33 | chr1p | TYW1 | chr7 |
| TMEM157 | chr5 | TRIM35 | chr8 | TYW3 | chr1p |
| TMEM161B | chr5 | TRIM36 | chr5 | UACA | chr15 |
| UBAP1 | chr9 | VAPA | chr18 | WIPI2 | chr7 |
| UBAP2L | chr1q/chr1q21 | VEZF1 | chr17 | WTAP | chr6q/chr6 |
| UBE2D4 | chr7 | VN1R1 | chr19 | XPA | chr9 |
| UBE2Q1 | chr1q/chr1q21 | VPS13A | chr9 | XPO4 | chr13 |
| UBE2Q2 | chr15 | VPS28 | chr8 | XPO5 | chr6 |
| UBE3A | chr15 | VPS36 | chr13 | XRCC4 | chr5 |
| UBL7 | chr15 | VPS37C | chr11 | YES1 | chr18 |
| UBLCP1 | chr5 | VPS4A | chr16 | YOD1 | chr1q |
| UBQLN4 | chr1q | VPS4B | chr18 | YTHDC2 | chr5 |
| UCHL3 | chr13 | VPS72 | chr1q/chr1q21 | YWHAZ | chr8 |
| UCK2 | chr1q | VPS8 | chr3 | YY1AP1 | chr1q |
| UFM1 | chr13 | VTI1B | chr14 | ZADH2 | chr18 |
| UGT2B17 | chr4 | WBP4 | chr13 | ZBTB2 | chr6q/chr6 |
| UHMK1 | chr1q | WDR20 | chr14 | ZBTB26 | chr9 |
| UHRF2 | chr9 | WDR21A | chr14 | ZBTB44 | chr11 |
| UIMC1 | chr5 | WDR22 | chr14 | ZBTB47 | chr3 |
| URG4 | chr7 | WDR23 | chr14 | ZBTB5 | chr9 |
| USP10 | chr16 | WDR32 | chr9 | ZC3H8 | chr2 |
| USP21 | chr1q | WDR36 | chr5 | ZC3HC1 | chr7 |
| USP25 | chr21 | WDR41 | chr5 | ZCCHC7 | chr9 |
| USP33 | chr1p | WDR47 | chr1p | ZDHHC23 | chr3 |
| USP4 | chr3 | WDR89 | chr14 | ZDHHC7 | chr16 |
| USPL1 | chr13 | WDSOF1 | chr8 | ZFP28 | chr19 |
| UTP14C | chr13 | WHSC1L1 | chr8 | ZFP3 | chr17 |
| ZFYVE21 | chr14 | | | | |
| ZMYM2 | chr13 | | | | |
| ZMYM5 | chr13 | | | | |
| ZNF16 | chr8 | | | | |
| ZNF184 | chr6 | | | | |
| ZNF193 | chr6 | | | | |
| ZNF195 | chr11 | | | | |
| ZNF20 | chr19 | | | | |
| ZNF230 | chr19 | | | | |
| ZNF236 | chr18 | | | | |
| ZNF257 | chr19 | | | | |
| ZNF259 | chr11 | | | | |
| ZNF311 | chr6 | | | | |
| ZNF313 | chr20 | | | | |
| ZNF337 | chr20 | | | | |
| ZNF346 | chr5 | | | | |
| ZNF395 | chr8 | | | | |
| ZNF416 | chr19 | | | | |
| ZNF434 | chr16 | | | | |
| ZNF439 | chr19 | | | | |
| ZNF442 | chr19 | | | | |
| ZNF443 | chr19 | | | | |
| ZNF498 | chr7 | | | | |
| ZNF557 | chr19 | | | | |

The reference cytogenetic abnormalities (rCA) of a given chromosome region were determined by the mean values of signals of aCGH probes located in that region. The cutoff value was set at 0.45 for amplification and −0.45 for deletion, as there were only 1% greater than 0.45 on the basis of the absolute signals of probes located in chromosomes 2, 4, 10, and 12, which are the most stable chromosomes in myeloma cells. The values of rCA could be used to distinguish among amplification, deletion, and normal. Reference values for different genomical regions are shown in Table 2.

TABLE 2
The cutoff values in the virtual CA
model for each location.

| Location | cutoff value |
|---|---|
| chr1p | 10.21 |
| chr6q | 10.36 |
| chr13 | 9.62 |
| chr1q21 | 10.17 |
| chr1q | 9.61 |
| chr3 | 9.42 |
| chr5 | 9.89 |
| chr7 | 9.18 |
| chr9 | 9.77 |
| chr11 | 9.95 |
| chr15 | 9.27 |
| chr19 | 7.75 |
| chr21 | 9.87 |

The predicted cytogenetic abnormalities (pCA) of a given chromosome region were determined by the following procedures. First, the mean expression levels of copy number-sensitive genes within the region were calculated. Then, by training the model in a gene expression profiling data set with 92 multiple myeloma samples, the cutoff value of the mean expression levels of copy number-sensitive genes for each chromosome region was set in order to obtain pCA that were most consistent with rCA in terms of the Matthews correlation coefficient, a measure of the quality of binary (two-class) classifications.

The mean prediction accuracy was 0.88 (0.59-0.99; Table 3 and Table 4) when the model was applied to the training data set. To check for overfitting in the vCA model, the model was applied to an independent data set of 23 multiple myeloma samples for which both gene expression profiling and aCGH data were available. The mean prediction accuracy was 0.89 (0.74-1.00; Table 3 and Table 5), which indicated that overfitting was negligible if present at all.

TABLE 3
Average prediction performances on different data sets

| Data Set | Sensitivity | Specificity | Accuracy |
|---|---|---|---|
| aCGH training set | 0.819 | 0.950 | 0.876 |
| aCGH test set | 0.881 | 0.908 | 0.893 |
| FISH | 0.883 | 0.876 | 0.874 |
| Karyotype | 0.705 | 0.632 | 0.648 |

TABLE 4
Prediction performance comparing vCA
model and aCGH in the training data set

| Location | Sensitivity | Specificity | Accuracy |
|---|---|---|---|
| chr1p | 0.710 | 0.918 | 0.848 |
| chr6q | 0.850 | 0.931 | 0.913 |
| chr13 | 0.768 | 0.972 | 0.848 |
| chr1q21 | 0.479 | 1.000 | 0.587 |
| chr1q | 0.897 | 0.962 | 0.935 |
| chr3 | 0.850 | 0.962 | 0.913 |
| chr5 | 0.973 | 1.000 | 0.989 |
| chr7 | 0.879 | 0.915 | 0.902 |
| chr9 | 0.909 | 0.973 | 0.935 |
| chr11 | 0.872 | 0.906 | 0.891 |
| chr15 | 0.923 | 0.975 | 0.946 |
| chr19 | 0.765 | 0.857 | 0.772 |
| chr21 | 0.774 | 0.984 | 0.913 |
| Mean | 0.819 | 0.950 | 0.876 |

TABLE 5
Prediction performance: vCA & aCGH in test set

| Location | Sensitivity | Specificity | Accuracy |
|---|---|---|---|
| chr1p | 1.000 | 1.000 | 1.000 |
| chr6q | 1.000 | 0.955 | 0.957 |
| chr13 | 0.900 | 1.000 | 0.957 |
| chr1q21 | 0.778 | 0.857 | 0.826 |
| chr1q | 0.750 | 0.867 | 0.826 |
| chr3 | 0.818 | 0.917 | 0.870 |
| chr5 | 0.909 | 1.000 | 0.957 |
| chr7 | 0.889 | 1.000 | 0.957 |
| chr9 | 1.000 | 0.909 | 0.957 |
| chr11 | 1.000 | 0.667 | 0.783 |
| chr15 | 0.923 | 1.000 | 0.957 |
| chr19 | 0.714 | 0.778 | 0.739 |
| chr21 | 0.778 | 0.857 | 0.826 |
| Mean | 0.881 | 0.908 | 0.893 |

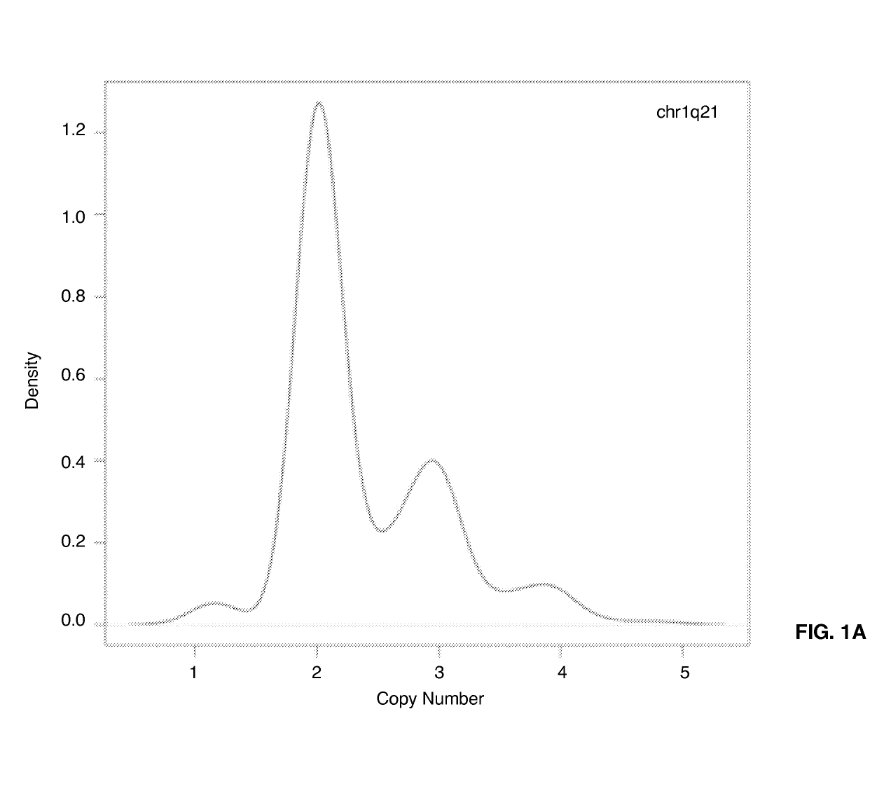

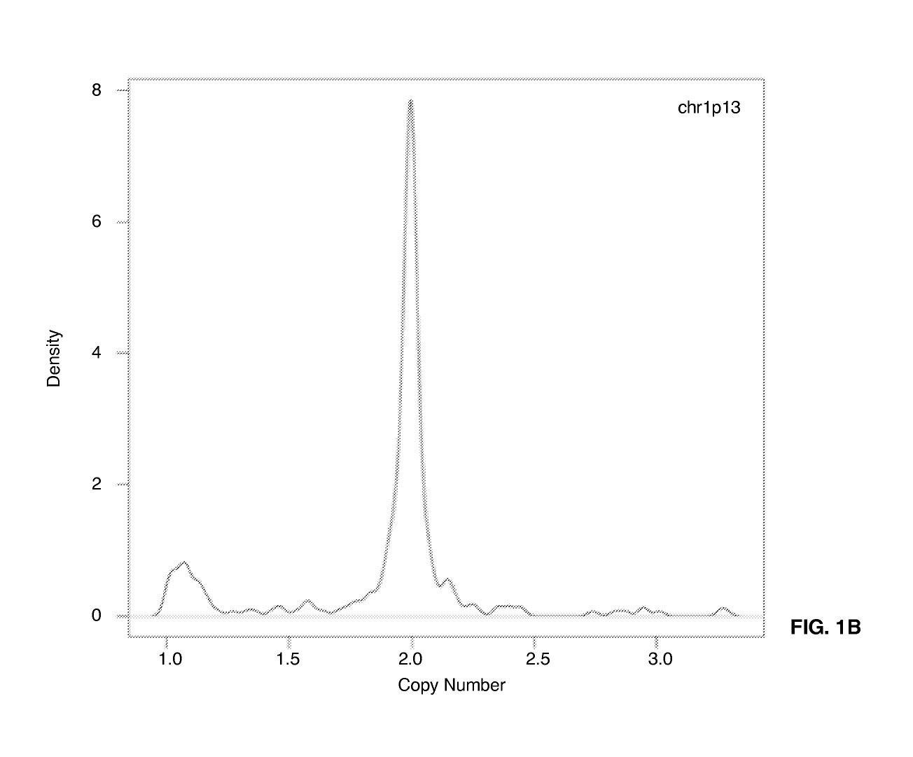

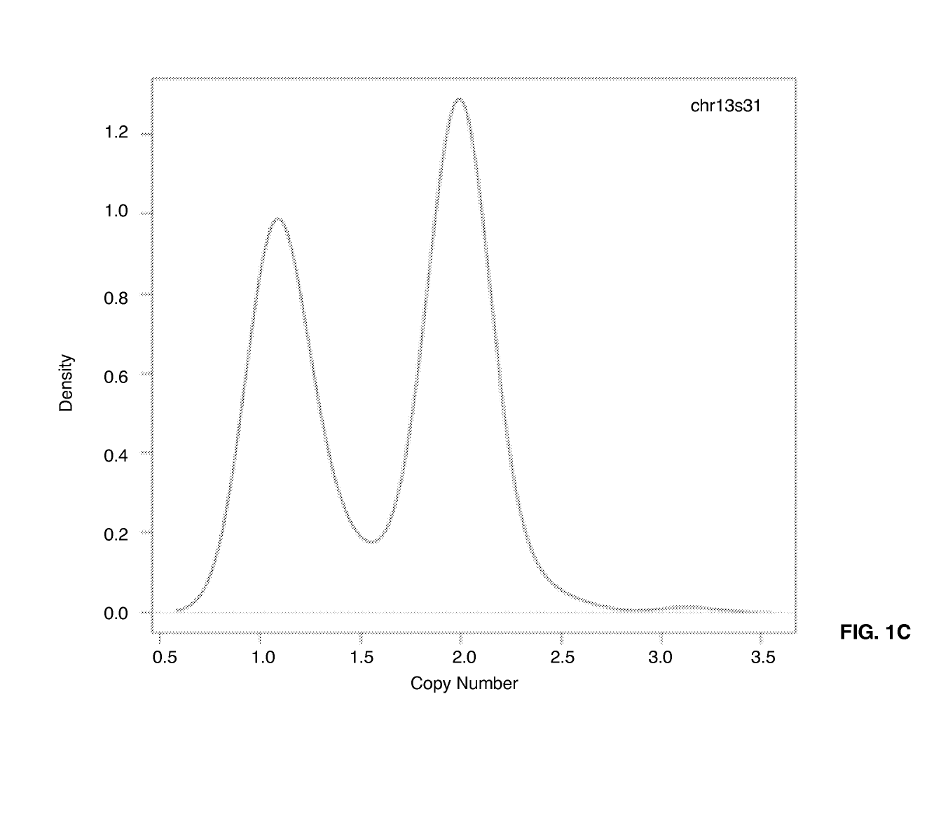

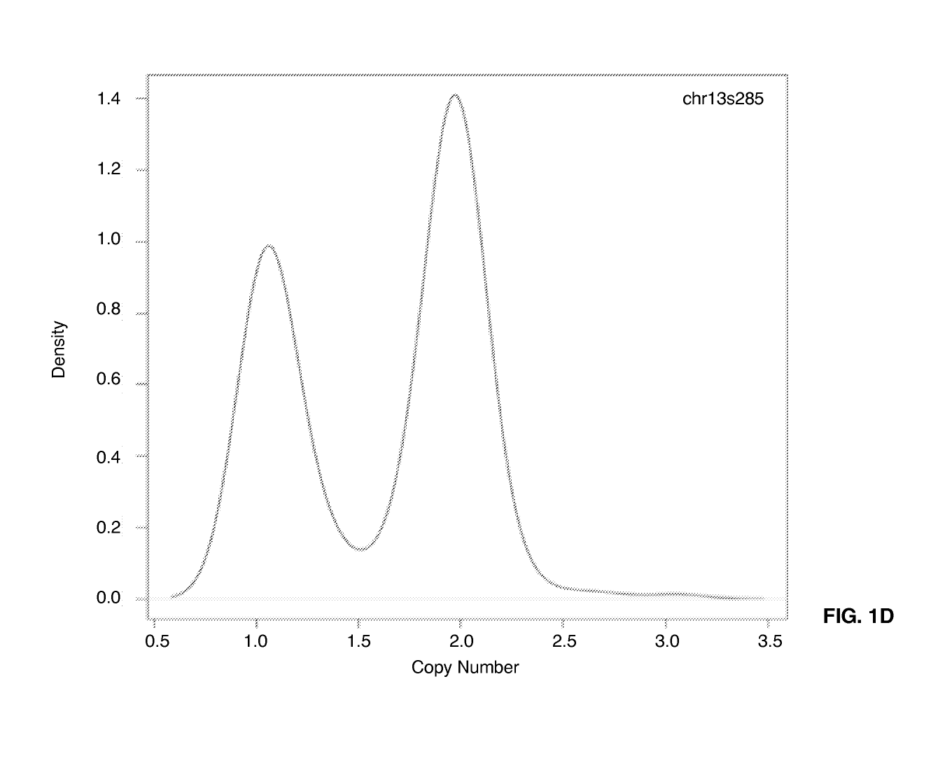

The model was validated with a FISH data set compiled from 262 independent MM samples for which both FISH records and GEP data were available. All 262 mM samples had been tested with 1p (AHCYL1) and 1q (CKS1B) probes. Of these samples, 195 had also been tested with chromosome 13 probes (D13S31 and D13S285). The cutoff value was set at 2.5 for amplification of 1q and at 1.5 for deletion of 1p and chr13, according to the distribution of the FISH signals (FIGS. 1A-1D). Applying the vCA model to the GEP data, we determined pCA for the 262 samples. The pCA results were well matched with the FISH reports. The mean prediction accuracy was 0.87 (0.82-0.90; Table 3 and Table 6).

TABLE 6
Prediction performance: vCA model and FISH reports

| Location | Sensitivity | Specificity | Accuracy |
|---|---|---|---|
| chr1q21 | 0.881 | 0.882 | 0.882 |
| chr1p13 | 0.882 | 0.811 | 0.821 |
| chr13s31 | 0.875 | 0.913 | 0.897 |
| chr13s285 | 0.895 | 0.899 | 0.897 |
| Mean | 0.883 | 0.876 | 0.874 |

In a further validation of the vCA model, a set of cytogenetic data was compiled which was generated by conventional karyotyping that included 533 independent multiple myeloma samples for which both karyotype records and GEP data were available. Applying the vCA model to the GEP data, the pCA was determined for the 533 samples. Although pCA results were matched to the karyotype reports with a mean prediction accuracy of 0.65 (0.36-0.77; Table 3 and Table 7), the consistency of the matching was lower than those of pCA vs. aCGH and pCA vs. FISH.

TABLE 7
Prediction performance comparing vCA
model and karyotype records

| | Sensitivity | Specificity | Accuracy |
|---|---|---|---|
| chr1p | 0.711 | 0.756 | 0.752 |
| chr1q | 0.835 | 0.712 | 0.732 |
| chr1q21 | 0.776 | 0.707 | 0.718 |
| chr3 | 0.688 | 0.662 | 0.665 |
| chr5 | 0.721 | 0.683 | 0.688 |
| chr6q | 0.475 | 0.771 | 0.749 |
| chr7 | 0.589 | 0.668 | 0.660 |
| chr9 | 0.806 | 0.468 | 0.527 |
| chr11 | 0.720 | 0.597 | 0.614 |
| chr13 | 0.663 | 0.630 | 0.635 |
| chr15 | 0.865 | 0.498 | 0.560 |
| chr19 | 0.849 | 0.260 | 0.355 |
| chr21 | 0.464 | 0.808 | 0.771 |
| Mean | 0.705 | 0.632 | 0.648 |

This prediction underperformance may be due to the fact that karyotyping can only detect the cytogenetic information for cells at metaphase, thus missing a considerable amount of information regarding the CN of DNA in a tumor cell population. If this is true, it would seem that FISH reports would also not match karyotype records well. To test this hypothesis, the FISH and karyotype data were compared for the 262 samples for which both records were available. Indeed, the prediction accuracies between FISH and karyotype records were 0.83, 0.76 and 0.60 for chr1p13, chr1q21 and chr13, respectively (Table 8), which is comparable to the prediction accuracies between pCA and karyotype (0.75, 0.72, 0.64 for chr1p13, chr1q21 and chr13, respectively; Table 7).

TABLE 8
Prediction performance comparing FISH reports and
karyotype records

| Location | Sensitivity | Specificity | Accuracy |
|---|---|---|---|
| chr1q21 | 0.855 | 0.736 | 0.759 |
| chr1p13 | 0.586 | 0.853 | 0.827 |
| chr13s31 | 0.714 | 0.573 | 0.599 |
| chr13s285 | 0.675 | 0.599 | 0.612 |
| Mean | 0.708 | 0.690 | 0.699 |

TABLE 9
Top 10 genes for each region
by correlation between gene
expression and aCGH.

| gene-name | correlation | location |
|---|---|---|
| ANP32E | 0.621921498 | chr1q |
| PMF1 | 0.61010205 | chr1q |
| CDC42SE1 | 0.604335048 | chr1q |
| CENPL | 0.596143746 | chr1q |
| NUF2 | 0.584414638 | chr1q |
| DARS2 | 0.579404421 | chr1q |
| SF3B4 | 0.577933484 | chr1q |
| PRKAB2 | 0.561313081 | chr1q |
| CKS1B | 0.55888504 | chr1q |
| RIT1 | 0.553182215 | chr1q |
| ANP32E | 0.621921498 | chr1q21 |
| CDC42SE1 | 0.604335048 | chr1q21 |
| SF3B4 | 0.577933484 | chr1q21 |
| PRKAB2 | 0.561313081 | chr1q21 |
| CKS1B | 0.55888504 | chr1q21 |
| ENSA | 0.545978858 | chr1q21 |
| IL6R | 0.537431607 | chr1q21 |
| CTSK | 0.534015087 | chr1q21 |
| VPS72 | 0.53337859 | chr1q21 |
| PRUNE | 0.529622458 | chr1q21 |
| WTAP | 0.585052819 | chr6q |
| REPS1 | 0.566167917 | chr6q |
| MAP3K4 | 0.534342516 | chr6q |
| TFB1M | 0.528556512 | chr6q |
| HDDC2 | 0.522301702 | chr6q |
| RWDD1 | 0.515964068 | chr6q |
| MTRF1L | 0.512760585 | chr6q |
| SYNCRIP | 0.508165214 | chr6q |
| HDAC2 | 0.505053284 | chr6q |
| PEX7 | 0.489761502 | chr6q |
| SIDT1 | 0.499036739 | chr3 |
| NR1I2 | 0.484486619 | chr3 |
| ZDHHC23 | 0.474793382 | chr3 |
| NISCH | 0.463271084 | chr3 |
| C3orf17 | 0.459054906 | chr3 |
| GTPBP8 | 0.455796834 | chr3 |
| KPNA1 | 0.450034074 | chr3 |
| EPHB1 | 0.447059932 | chr3 |
| MRPS25 | 0.436842545 | chr3 |
| IRAK2 | 0.43495804 | chr3 |
| F2R | 0.576371069 | chr5 |
| ELOVL7 | 0.550513362 | chr5 |
| THG1L | 0.54860992 | chr5 |
| ADAM19 | 0.535568989 | chr5 |
| BNIP1 | 0.507497946 | chr5 |
| UBLCP1 | 0.501918885 | chr5 |
| EPB41L4A | 0.499599416 | chr5 |
| TCOF1 | 0.497784224 | chr5 |
| HDAC3 | 0.487597992 | chr5 |
| TMEM161B | 0.470891239 | chr5 |
| SMURF1 | 0.488174377 | chr7 |
| C7orf46 | 0.459221625 | chr7 |
| UBE2D4 | 0.451083252 | chr7 |
| GNG11 | 0.447485478 | chr7 |
| WIPI2 | 0.446328202 | chr7 |
| PHF14 | 0.441814806 | chr7 |
| LSM5 | 0.439762406 | chr7 |
| TYW1 | 0.431604316 | chr7 |
| C7orf41 | 0.424046711 | chr7 |
| EGFR | 0.410176459 | chr7 |
| RALGPS1 | 0.606835402 | chr9 |
| TBC1D13 | 0.569804522 | chr9 |
| UBAP1 | 0.549886963 | chr9 |
| NIPSNAP3B | 0.517057023 | chr9 |
| BAG1 | 0.511360495 | chr9 |
| WDR32 | 0.500472126 | chr9 |
| ZBTB26 | 0.500380065 | chr9 |
| GARNL3 | 0.492871978 | chr9 |
| ANKRD15 | 0.477440514 | chr9 |
| RNF38 | 0.450522342 | chr9 |
| BIRC2 | 0.773828195 | chr11 |
| TMEM123 | 0.766964978 | chr11 |
| TMEM133 | 0.548926336 | chr11 |
| FCHSD2 | 0.52452816 | chr11 |
| NPAT | 0.514283073 | chr11 |
| RAB1B | 0.510430279 | chr11 |
| PAK1 | 0.505182294 | chr11 |
| DCUN1D5 | 0.50141626 | chr11 |
| ANKRD49 | 0.500386277 | chr11 |
| SAAL1 | 0.499245319 | chr11 |
| USPL1 | 0.698412782 | chr13 |
| PSPC1 | 0.696853829 | chr13 |
| SAP18 | 0.636296236 | chr13 |
| STK24 | 0.626179693 | chr13 |
| XPO4 | 0.62611934 | chr13 |
| TGDS | 0.601638669 | chr13 |
| MYCBP2 | 0.59897856 | chr13 |
| MRPS31 | 0.596652017 | chr13 |
| PCID2 | 0.589548383 | chr13 |
| NUFIP1 | 0.585274816 | chr13 |
| CEP27 | 0.58744229 | chr15 |
| PML | 0.525229128 | chr15 |
| ABHD2 | 0.495682942 | chr15 |
| LRRC57 | 0.492887584 | chr15 |
| ISL2 | 0.477106522 | chr15 |
| DENND4A | 0.471341444 | chr15 |
| C15orf17 | 0.469029084 | chr15 |
| C15orf40 | 0.464802307 | chr15 |
| EDC3 | 0.45645991 | chr15 |
| AVEN | 0.453069349 | chr15 |
| KLHL26 | 0.516147054 | chr19 |
| CCNE1 | 0.502666127 | chr19 |
| OPA3 | 0.493457802 | chr19 |
| ZNF442 | 0.485749329 | chr19 |
| VN1R1 | 0.47250557 | chr19 |
| DENND1C | 0.472265334 | chr19 |
| ZNF20 | 0.471146644 | chr19 |
| ZNF230 | 0.464598815 | chr19 |
| DMPK | 0.452919613 | chr19 |
| OR7A5 | 0.436401136 | chr19 |
| DSCR3 | 0.504339046 | chr21 |
| AGPAT3 | 0.495164723 | chr21 |
| PCNT | 0.470010827 | chr21 |
| SETD4 | 0.46765593 | chr21 |
| BRWD1 | 0.448381222 | chr21 |
| IFNGR2 | 0.439633799 | chr21 |
| TMEM1 | 0.41910999 | chr21 |
| IL10RB | 0.417444441 | chr21 |
| C21orf33 | 0.408839039 | chr21 |
| CHODL | 0.393694133 | chr21 |
| GTF2B | 0.638320526 | chr1p |
| TRIM33 | 0.620456081 | chr1p |
| CSDE1 | 0.555728605 | chr1p |
| CEPT1 | 0.55400251 | chr1p |
| EVI5 | 0.539604672 | chr1p |
| LMO4 | 0.517238178 | chr1p |
| SH3GLB1 | 0.504284974 | chr1p |
| RWDD3 | 0.502570278 | chr1p |
| PKN2 | 0.492688787 | chr1p |
| AGL | 0.491653201 | chr1p |

### REFERENCES

1. Barlogie B, Epstein J, Sanderson R, Anaissie E, Walker R, Tricot G. Plasma cell myeloma. In: Lichtman M A, Kaushansky K, Kipps T J, Seligsohn U, Prchal J, eds. Williams Hematology (7th Ed). New York, N.Y.: McGraw-Hill; 2005:1501-1533.
2. Kuehl W M, Bergsagel P L. Multiple myeloma: evolving genetic events and host interactions. Nat Rev Cancer. 2002; 2(3):175-187.
3. Seong C, Delasalle K, Hayes K, et al. Prognostic value of cytogenetics in multiple myeloma. Br J Haematol. 1998; 101(1):189-194.
4. Stewart A K, Fonseca R. Prognostic and therapeutic significance of myeloma genetics and gene expression profiling. J Clin Oncol. 2005; 23(26):6339-6344.
5. Zhan F, Hardin J, Kordsmeier B, et al. Global gene expression profiling of multiple myeloma, monoclonal gammopathy of undetermined significance, and normal bone marrow plasma cells. Blood. 2002; 99(5):1745-1757.

6. Shaughnessy J, Tian E, Sawyer J, et al. High incidence of chromosome 13 deletion in multiple myeloma detected by multiprobe interphase FISH. Blood. 2000; 96(4):1505-1511.
7. Sawyer J R, Waldron J A, Jagannath S, Barlogie B. Cytogenetic findings in 200 patients with multiple myeloma. Cancer Genet Cytogenet. 1995; 82(1): 41-49.
8. Zhan F, Huang Y, Colla S, et al. The molecular classification of multiple myeloma. Blood. 2006; 108(6):2020-2028.
9. Shaughnessy J D Jr, Zhan F, Burington B E, et al. A validated gene expression model of high-risk multiple myeloma is defined by deregulated expression of genes mapping to chromosome 1. Blood. 2007; 109(6):2276-2284.
10. Zhan F, Barlogie B, Arzoumanian V, et al. Geneexpression signature of benign monoclonal gammopathy evident in multiple myeloma is linked to good prognosis. Blood. 2007; 109(4):1692-1700.
11. Yang Y H, Dudoit S, Luu P, et al. Normalization for cDNA microarray data: a robust composite method addressing single and multiple slide systematic variation. Nucleic Acids Res. 2002; 30(4): e15.
12. Venkatraman E S, Olshen A B. A faster circular binary segmentation algorithm for the analysis of array CGH data. Bioinformatics. 2007; 23(6):657-663.
13. Baldi P, Brunak S, Chauvin Y, Andersen C A, Nielsen H. Assessing the accuracy of prediction algorithms for classification: an overview. Bioinformatics. 2000; 16(5):412-424.
14. Chang H, Qi C, Yi Q L, Reece D, Stewart A K. p53 gene deletion detected by fluorescence in situ hybridization is an adverse prognostic factor for patients with multiple myeloma following autologous stem cell transplantation. Blood. 2005; 105(1):358-360.
15. Neben K, Lokhorst H M, Jauch A, et al. Administration of bortezomib before and after autologous stem cell transplantation improves outcome in multiple myeloma patients with deletion 17p. Blood. 2012; 119(4):940-948.
16. Chapman M A, Lawrence M S, Keats J J, et al. Initial genome sequencing and analysis of multiple myeloma. Nature. 2011; 471(7339):467-472.

Any patents or publications mentioned in this specification are indicative of the levels of those skilled in the art to which the invention pertains. These patents and publications are incorporated by reference herein to the same extent as if each individual publication was incorporated by reference specifically and individually.

One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control.

Headings used in this application are for convenience only and do not affect the interpretation of this application.

While this invention has been particularly shown and described with references to example embodiments thereof, it will be understood by those skilled in the art that various changes in form and details may be made therein without departing from the scope of the invention encompassed by the appended claims.

What is claimed is:

1. A method for predicting the presence of a cytogenetic abnormality located in a chromosomal region and associated with multiple myeloma in a subject, comprising testing the gene expression level of a set of the copy number sensitive genes of Table 1 located in the chromosomal region in cells isolated from the subject, wherein abnormal gene expression levels of the copy number sensitive genes, relative to a suitable control, indicates the presence of a cytogenetic abnormality selected from a gain of chr1q, chr3, chr5, chr7, chr9, chr11, chr15, chr19, or chr21; amplification of chr1q21; or loss of chr1p, chr6q, or chr13q.

2. The method of claim 1, wherein the cells are plasma cells.

3. The method of claim 2, wherein the plasma cells are CD138-enriched.

4. The method of claim 1, wherein the gene expression levels are determined by Southern blotting, Northern blotting, microarray, real-time polymerase chain reaction (PCR) (RT-PCR), quantitative PCR (qPCR), qRT-PCR, or nucleic acid sequencing.

5. The method of claim 4, wherein the microarray is a DASL Human Cancer Panel, DASL custom array, U133, U133A 2.0, or U133 Plus 2.0 array.

6. The method of claim 4, wherein the sequencing is whole transcriptome shotgun sequencing (RNA-seq), sequencing by synthesis, pyrosequencing, dideoxy sequencing, or sequencing by ligation.

7. The method of claim 1, further comprising testing the TP53 status of the subject by gene expression profiling.

8. The method of claim 7, wherein the TP53 gene expression profiling comprises testing the level of gene expression of the TP53-regulated genes TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD2A, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B, SEPP1, and C18orf54 in plasma cells from the individual; and
assigning the individual a classification after comparing the expression level of the genes with the expression level of the genes in one or more controls with a high or low level of TP53 gene expression, wherein a low level of TP53 gene expression is associated with a poor prognosis, wherein
a) decreased expression of one or more of ABCB9, AGRN, ALOX5, ANKRA2, APP, ARHGAP25, BTG2, C14orf28, C18orf1, CENTD2, COPG, CYB5D2, DLC1, ECHDC2, FKSG44, FXYD5, GAA, HDLBP, HIGD2A, IFIT5, KCNS3, KLHL24, LAPTM4B, LOC387763, MAN1C1, MCC, MKNK2, NADSYN1, NCKAP1, NOTCH1, OSBPL10, PHLDB1, RAB1A, RTN2, SAT2, SESN1, SIDT1, SLAMF7, STAT3, STT3B, TM7SF2, TMEM57, TncRNA, TNFRSF10B, TRIM13, TRIM22, UNC93B1, WNT16, ZMAT3, and ZNF600 is associated with a low level of TP53 gene expression; and
b) increased expression of one or more of ANLN, ASPM, B3GALNT1, BRCA1, C18orf54, C5orf34, CCNE2, CD302, CDC25A, CDCA2, CDCA7, CEP55, DCUN1D4, DEPDC1, ENPP4, FAM111B, FANCI, HFE, INTS7, KIF2C, L3 MBTL4, MEIS1, NEIL3, OPN3, PYGL, RIF1, RIT1, S100A4, SEPP1, SGOL2, SLC4A7, SPRED1, SUV39H2, THEX1, and TUBA1A is associated with a low level of TP53 gene expression.

9. The method of claim 1, wherein the gene expression levels of the copy number sensitive genes of Table 1 located in the chromosomal region are evaluated against threshold values substantially similar to those in Table 2.

10. The method of claim 1, further comprising testing the GEP-17, GEP-70, or GEP-80 profile for the subject.

11. The method of claim 1 wherein the subject has multiple myeloma, smoldering myeloma, or monoclonal gammopathy of undetermined significance (MGUS).

12. The method of claim 11, wherein the subject is undergoing treatment with chemotherapy, hormonal therapy, immunotherapy, radiotherapy, or a combination thereof.

13. The method of claim 12, wherein the subject is undergoing treatment comprising bortezomib.

14. The method of claim 12, wherein the subject is undergoing total therapy 2 treatment.

15. The method of claim 12, wherein the subject is undergoing total therapy 3 treatment.

16. A non-transitory computer-readable storage medium that provides instructions that, if executed by a computer, will cause the computer to perform operations comprising
comparing the gene expression level of a set of the copy number sensitive genes of Table 1 located in a chromosomal region in cells isolated from a subject to suitable control values; and
outputting a value predictive of one or more cytogenetic abnormalities in the chromosomal region selected from a gain of chr1q, chr3, chr5, chr7, chr9, chr11, chr15, chr19, or chr21; amplification of chr1q21; or loss of chr1p, chr6q, or chr13q in the subject based on the comparison,
wherein abnormal gene expression levels of the set of copy number sensitive genes of Table 1 located in the chromosomal region, relative to the suitable control values, indicates the presence of one or more of the cytogenetic abnormalities.

17. A computer comprising the storage medium of claim 16 and a processor for executing the instructions.

18. The computer of claim 17, further comprising an input means adapted to receive gene expression values for the copy number sensitive genes of Table 1 located in the particular chromosomal region for the cells isolated from the subject.

19. A method for predicting the presence of a cytogenetic abnormality located in a chromosomal region in the absence of FISH (fluorescent in situ hybridization) analysis, the cytogenetic abnormality selected from a gain of chr1q, chr3, chr5, chr7, chr9, chr11, chr15, chr19, or chr21; amplification of chr1q21; or loss of chr1p, chr6q, or chr13q in a subject having multiple myeloma, comprising inputting gene expression levels of a set of the copy number sensitive genes of Table 1 located in the chromosomal region, in cells isolated from the subject, into the computer of claim 18, executing the program instructions, and obtaining the outputted value predictive of the cytogenetic abnormalities in the subject.

20. The method of claim 1, wherein a gain of chr1q, chr3, chr5, chr7, chr9, chr11, chr15, chr19, and chr21; amplification of chr1q21; and loss of chr1p, chr6q, and chr13q is detected.

21. The methods of claim 1, wherein the method is performed in the absence of FISH analysis.
